(19) United States
(12) Patent Application Publication
(10) Pub. No.: US 2011/0009909 A1
(43) Pub. Date: Jan. 13, 2011

(54) OSTEOSYNTHESIS DEVICE COMPRISING A SUPPORT WITH A TAPPED ORIFICE ASSOCIATED WITH A BEARING SURFACE FOR RECEIVING AN ANCHORING ROD

(75) Inventor: Guillaume Derouet, Saint-Herblain (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: D.L.P., La Haye Fouassiere (FR)

(21) Appl. No.: 12/521,388

(22) PCT Filed: Dec. 26, 2007
(86) PCT No.: PCT/FR2007/052624

§ 371 Date: Jun. 26, 2009

(30) Foreign Application Priority Data
Dec. 27, 2006 (FR) 0611444
Dec. 27, 2006 (FR) 0611445
Dec. 27, 2006 (FR) 0611447
Dec. 27, 2006 (FR) 0611449

(51) Int. Cl. A61B 17/86

(52) U.S. Cl. 606/305; 606/315

(57) ABSTRACT

This osteosynthesis device is of the type comprising: a support provided with a through-orifice, at least one part of the height of which is threaded, and an anchoring rod having a threaded part forming a screw, the thread of said threaded part being complementary to that of said support orifice, in order to hold said rod on said support by screwing, said support and said rod furthermore including complementary bearing surfaces. The protruding ends of the screw threads and support threads are truncated in such a way that the difference between the outside screw diameter and the flank diameter is between 5 and 70% of the difference between the nominal diameter and the flank diameter, and the difference between the flank diameter Df and the core diameter is between 5 and 70% of the difference between the flank diameter and the root diameter.

The present invention relates to the field of devices for osteosynthesis techniques; more particularly, it relates to osteosynthesis devices of the type comprising a support provided with at least one tapped orifice associated with a bearing surface for receiving an anchoring rod, said rod having a head extended by a body adapted to anchor into the receiving bone material, wherein said head comprises a screw threaded part and a bearing surface, the thread of said threaded part being complementary to that of said support orifice so as to hold said rod on said support by screwing, and said support and rod head bearing surfaces being complementary to one another and arranged so as to come into contact with one another when said complementary threads cooperate with each other.

The bone consolidation of certain fractures needs osteosynthesis equipments or devices to be mounted on the different bone fragments, to stabilize and prevent an excessive inter-fragment mobility.

The practitioners then use osteosynthesis devices screwed or forced into the bone fragments, such as screws, pins or screwed plates, so as to apply a determined and appropriate compression to the bone.

Some of such devices are composed of a support plate provided with one or several orifices, that is associated with one or several rods each inserted into one of said orifices and intended to anchor into the receiving bone. The anchoring rod(s) comprise a generally threaded body for anchoring into the bone, and a head upper part adapted to cooperate with the support plate.

In some cases, in particular when a mono-axial locking is desired, this rod head comprises a thread adapted to cooperate with a complementary thread provided in the receiving orifice of the support plate. Further, complementary bearing surfaces provided both on the support and on the rod head ensure the locking of the rod progression into the receiving bone material.

Such structures are described for example in FR-742 618 or in U.S. Pat. No. 5,085,660.

But, in this technical field, the size of the different pieces is minimized, in particular to limit the implantation traumatism, and the rod and support threads are very small.

It is therefore necessary to make sure that the rod is introduced in the axis of the support orifice to ensure a correct cooperation between the threads, so as to avoid generating in the contact areas stresses that make the screwing difficult and that might bring about deformations and deteriorations of the threads. Thus, the current materials do not or almost not tolerate any coaxiality defect between the rod and the axis of the support orifice, which is harmful to easiness and comfort of installation.

To remedy these drawbacks, the present invention proposes to truncate the protruding ends of said screw threads (threaded part of the rod) and support threads in a much more substantial manner than the conventional functional truncations. More precisely, taking into account the classical values of diameter for defining a screw cooperating with a tapped orifice provided in a support (nominal diameter Dn and flank diameter Df for the two elements; root diameter Dff and outside diameter De for the screw; core diameter Dns for the support), the corresponding truncation is as follows:
the difference Δtv between the outside screw diameter De and the flank diameter Df is between 5 and 70% of the difference Δsv between the nominal diameter Dn and the flank diameter Df, and
the difference Δts between the flank diameter Df and the core diameter Dns is between 5 and 70% of the difference Δss between the flank diameter Df and the root diameter Dff.

Preferably, Δtv is between 15 and 50% of Δsv, and even more preferably Δtv is about 20% of Δsv. On the other hand, Δts is preferably between 8 and 50% of Δss, with a preferential value about 10%.

The corresponding substantial truncation of the protruding ends of the screw and support threads allows an easier meshing of said threads at the time of screwing, while improving the robustness of said threads.

A tolerant system that allows a small margin of error as for the coaxiality at the beginning of screw introduction into the support is obtained. Moreover, the mechanical strength quality of the system screw/support is absolutely not handicapped, the stress transfer being ensured at the flank diameter.

The complementary threads of the support and the rod head may be single-, double- or triple-threads, or even more.

According to a particular embodiment, the support and rod head bearing surfaces are in the form of a truncated sphere. The truncated-sphere bearing surface of the support is provided at the exit of the orifice threaded part; and the truncated-sphere bearing surface of the rod head is provided under the screw threaded part in the continuation of the latter.

Within this framework, the through-orifice of the support advantageously comprises (taking into account the direction of introduction of the rod) a threaded upper part, located above a non-threaded lower part defining the truncated-sphere bearing surface.

According to another particular embodiment, the support and rod head bearing surfaces are in the form of a truncated cone. The truncated-cone bearing surface of the support is provided above the tapped orifice; and the truncated-cone bearing surface of the rod head is provided above the screw threaded part.

Within this framework, the through-orifice of the support advantageously comprises (taking into account the direction of introduction of the rod) a non-threaded upper part, located above the threaded part, said orifice upper part comprising the truncated-cone bearing surface and ensuring the complete integration of the rod head upper part, once the rod correctly anchored in the receiving bone material.

Still within this framework, the support advantageously comprises a reentrant in the form of a spherical bearing surface, centred on the orifice axis, provided between the truncated-cone bearing surface and the threaded part of said orifice. If need be, the support may then receive, in replacement of the aforesaid mono-axial locking anchoring rod, a poly-axial screw whose head comprises a spherical bearing which is complementary to said support spherical bearing.

According to still another possible embodiment, the support and rod head bearing surfaces are plane surfaces. The plane bearing surface of the support is provided at the entrance of the orifice threaded part; and the plane bearing surface of the rod head is provided above the screw-threaded part, said plane bearing surface of the rod head defining the lower face of a head upper base.

Within this framework, the through-orifice of the support advantageously comprises (taking into account the direction of introduction of the rod) a non-threaded upper part, located above the threaded part and separated from the latter by the aforesaid plane bearing surface, said orifice upper part ensuring the complete integration of the rod head upper base, once the rod correctly anchored in the receiving bone material.

The support advantageously consists of a metal plate having a thickness between 1 and 5 mm.

The invention will be further illustrated, without being in any way limited, by the following description associated with the appended drawings, in which:

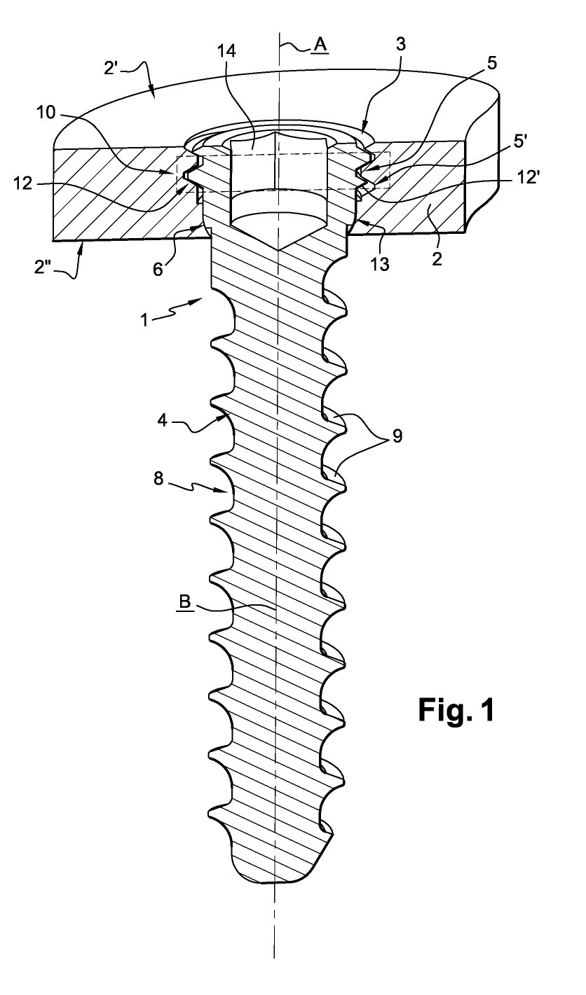

FIG. 1 is a schematic cross-sectional view of an osteosynthesis device according to the present invention, comprising a support provided with a tapped orifice associated with a truncated-sphere bearing and an anchoring rod (herein in the form of an anchoring screw);

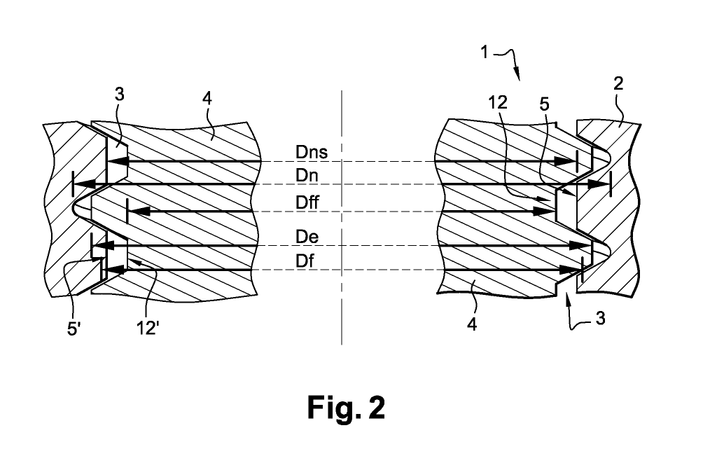

FIG. 2 is an enlarged view of a portion of FIG. 1, showing in detail the structure of the rod and support complementary threads;

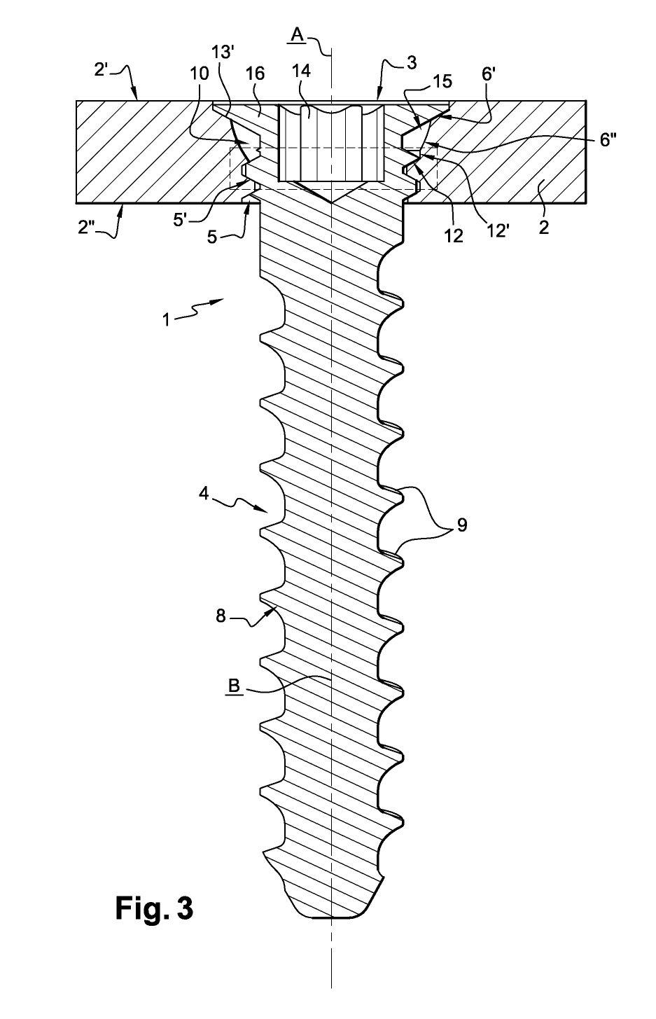

FIG. 3 is a schematic cross-sectional view of another embodiment of an osteosynthesis device according to the present invention, comprising a support provided with a tapped orifice associated with a truncated-cone bearing and an anchoring rod (herein in the form of an anchoring screw);

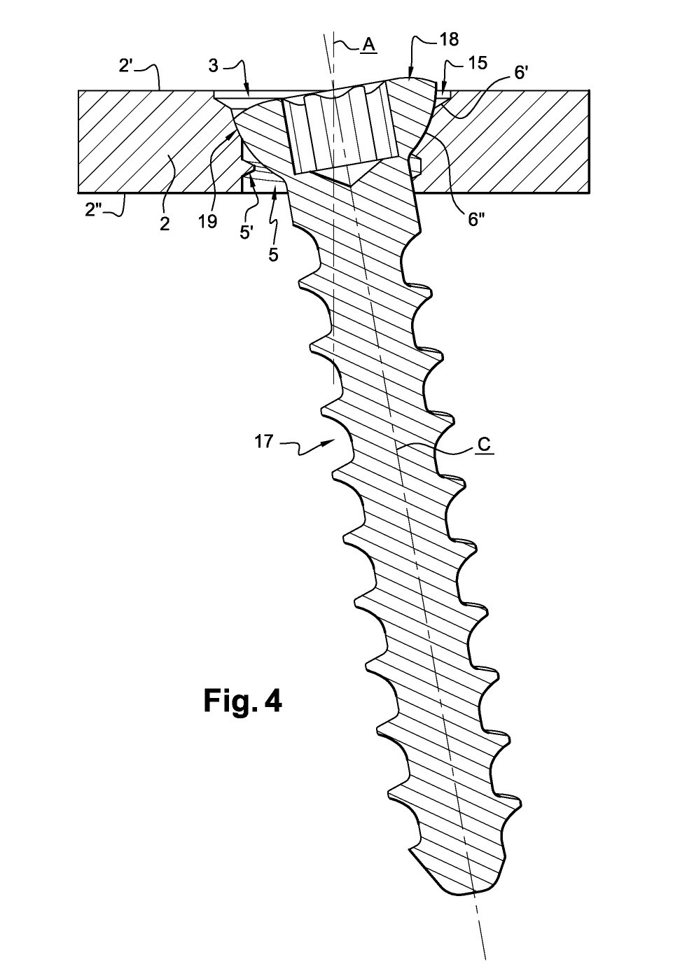

FIG. 4 shows the association of the support of FIG. 3 with a poly-axial-type screw;

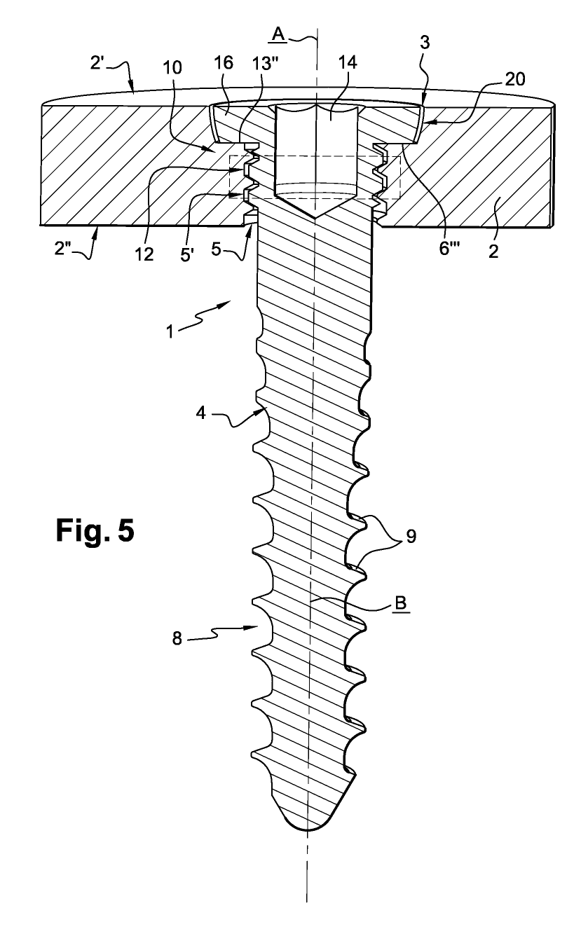

FIG. 5 is a schematic cross-sectional view of still another embodiment of an osteosynthesis device according to the present invention, comprising a support provided with a tapped orifice associated with a plane bearing and an anchoring rod (herein in the form of an anchoring screw).

As schematically illustrated in FIGS. 1 and 2, the osteosynthesis device 1 comprises a support element 2 provided with a through-orifice 3, for receiving a rod 4, one end of which is adapted to anchor into a receiving bone material.

The support 2 can take any possible form. As illustrated in FIG. 1, it is preferably a thin metal plate (for example, 1 to 5 mm thick), having an upper face 2′ and a lower face 2″, the latter being intended to come into contact with the bone material (not shown).

The orifice 3 for receiving the rod 4 is formed of a part 5 provided with a thread 5′ of axis A ending at the upper face 2′ of the support 2, extended by a non-threaded part 6, in the form of a truncated sphere, centred on said axis A, which ends at the lower face 2″ of the support 2. The bearing surface 6 is provided just at the exit of the tapped part 5.

The rod 4 of axis B comprises an end body 8 provided with a thread 9 intended to anchor into the receiving bone material. Its other end comprises a head 10 formed of an upper threaded cylindrical part 12, extended toward the end body 8 by a non-threaded part 13 forming a truncated-sphere bearing surface centred on the axis B of the rod 4. This truncated-sphere bearing surface 13 is provided directly under the threaded part 12.

The thread 12′ of the threaded part 12 is complementary to the support thread 5′ so as to hold the rod 4 on its support 2 by screwing. Said thread 12′ may have several turns.

In FIG. 1, it can be seen that the rod head 10 comprises an axial indentation 14, in the form of a hexagonal orifice, for example, intended for the screwing (and possibly unscrewing) operations using a suitable tool.

In another embodiment, the end body 8 of the rod 4 may have a smooth external surface, with no thread.

As said above, the present invention relates to the particular structure of the complementary support and rod threads 5′, 12′, and more particularly to the substantial truncation of the protruding ends of said threads. Said threads 5′, 12′ have standard envelops. Said threads 5′, 12′ do not have any diminution between the flanks with respect to the conventional threads; their novelty lies only in the substantial truncation of their protruding ends.

As illustrated in FIG. 2:

Dn corresponds to the nominal diameter of the system screw/support, i.e. the nominal diameter of the tapped cylindrical part 5 of the orifice 3 and that of the threaded part 12 of the rod 4;

Df corresponds to the flank diameter of the system screw/support, i.e. the flank diameter of the tapped cylindrical part 5 of the orifice 3 and that of the threaded part 12 of the rod 4;

Dff corresponds to the root diameter of the threaded part 12 of the rod 4;

Dns corresponds to the core diameter of the threaded part 5 of the orifice 3 of the support 2; and

De is the outside diameter of the threaded part 12 of the rod 4.

According to the invention, if:
Δtv corresponds to the difference between De and Df,
Δsv is the difference between Dn and Df,
Δts is the difference between Df and Dns, and
Δss is the difference between Df and Dff,

then: Δtv=5 to 70% (preferably 15 to 50%) of Δsv

and Δts=5 to 70% (preferably 8 to 50%) of Δss.

The preferred values of Δtv and Δts are about 20% of Δsv and 10% of Δss, respectively.

The threads 5′ and 12′ of the osteosynthesis device illustrated in FIGS. 1 and 2 are single-threads. To further facilitate insertion of the threaded part 12 of the rod 4 into the tapped hole 5, said threads may be double-threads, triple-threads, or even have more than three threads.

Conventionally, the support element 2 may comprise a plurality of tapped orifices 3 each receiving an anchoring rod 4.

As illustrated in FIG. 1, at the end of screwing, the truncated-sphere bearing surface 13 of the screw head 10 comes into spherical contact with the bearing surface 6 of the support 2. This spherical contact has interesting contact-surface and compactness qualities.

In another possible embodiment, the surfaces 6, 13 for spherical contact between the rod 4 and the support 2 may be provided above the complementary threaded parts 5 and 12. In this case, the bearing surface 6 of the support 2 is advantageously provided at the entrance of the threaded part 5.

FIG. 3 illustrates another embodiment of an osteosynthesis device according to the invention.

For the sake of simplifying the description, the parts that are similar to those of the device illustrated in FIGS. 1 and 2 are denoted by the same reference numbers.

Herein, the orifice 3 for receiving the rod 4 is formed of a part 5 provided with a thread 5′ of axis A above which is located a non-threaded part 15. Said non-threaded part 15 whose diameter is greater than that of the threaded part 5 ends at the upper face 2′ of the support plate 2; it comprises an upper part 6′ (ending at the upper face 2′ of the support 2) which is herein in the form of a truncated-cone bearing surface extended, on the side of the threaded part 5, by a reentrant 6″ in the form of a spherical bearing surface.

The truncated-cone bearing surface 6′ is thus located above the threaded part 5 of the orifice 3, being simply separated from the entrance of the latter by the reentrant 6″, and it is centred on the axis A of the orifice 3. The threaded part 5 ends at the lower face 2″ of the support 2.

The spherical bearing surface 6″ is also centred on the axis A of the orifice 3.

The rod 4 of axis B comprises an end body 8 provided with a thread 9 intended to anchor into the receiving bone material. Its other end comprises a head 10 formed of an upper base 16 connected to an underlying threaded part 12 having a smaller diameter. The base 16 of the rod head 10 may have a generally circular shape; it comprises a lower bearing surface 13′, herein in the general form of a truncated cone, which is intended to come into plane contact with the aforesaid truncated-cone bearing surface 6′ of the support 2. Said bearing surface 13′ is centred on the axis B of the rod 4.

Again, the thread 12′ of the threaded part 12 is complementary to the support thread 5′ so as to hold the rod 4 on its support 2 by screwing; furthermore, said threads 5′ and 12′ comprise truncated protruding ends, as described above with reference to FIG. 2.

As illustrated in FIG. 3, at the end of screwing, the truncated-cone bearing surface 13′ of the rod head 10 comes into conical contact with the bearing surface 6′ of the support 2; moreover, the base 16 of said rod head 10 is completely integrated in the non-threaded part 15 of the support 2 (in this position, the upper part of the rod head 10 makes flush with the upper face 2′ of the support 2).

This conical contact has interesting contact-surface and compactness qualities.

In another possible embodiment, the surfaces 6′, 13′ for conical contact between the rod 4 and the support 2 may be provided under the complementary threaded parts 5 and 12. In this case, the bearing surface 6′ of the support 2 is advantageously provided at the exit of the threaded part 5.

As illustrated in FIG. 4, if the practitioner wants so, the orifice 3 of the support 2 may be equipped, in replacement of the mono-axial locking anchoring rod 4, with a poly-axial screw 17 whose head 18 is provided with a spherical bearing surface 19 complementary to the support spherical bearing 6″.

Said poly-axial screw 17 does not comprise a thread liable to cooperate with the orifice thread 5′ of the support 2 and is therefore not locked. It enables the practitioner to implement screw orientations that are different from those imposed by the above-described locking system (it will be noticed that axis C of the screw 17 is not merged with the orifice axis A in FIG. 4), notably to generate return effects on bone fragments upon the compression bearing of the rod head 19 against the support 2 (through the spherical bearing 6″).

FIG. 5 illustrates still another embodiment of an osteosynthesis device according to the invention.

Again, the parts that are similar to those of the devices illustrated in FIGS. 1 to 4 are denoted by the same reference numbers.

Herein, the orifice 3 for receiving the rod 4 is formed of a part 5 provided with a thread 5′ of axis A, above which is located a non-threaded part 20. Said non-threaded part 20 whose diameter is greater than that of the threaded part 5 ends at the upper face 2′ of the support plate 2 and connects to said threaded part 5 by a plane bearing surface 6′″, which is generally annular and which is perpendicular to the axis A of the orifice 3. The plane bearing surface 6′″ is provided at the entrance of the threaded part 5 of the orifice 3; said threaded part 5 ends at the lower face 2″ of the support 2.

The rod 4 comprises an end body 8 provided with a thread 9 intended to anchor into the receiving bone material. Its other end comprises a head 10 formed of an upper base 16 connected to an underlying threaded part 12 having a smaller diameter. The base 16 of the rod head 10 may have a generally circular shape; it comprises a lower bearing surface 13′ which ensures the connexion thereof with the threaded part 12 and which is perpendicular to the axis B of the rod 4. Such bearing surface 13″ is intended to come into plane contact with the aforesaid bearing surface 6″ of the support 2.

Again, the thread 12′ of the threaded part 12 is complementary to the support thread 5′ so as to hold the rod 4 on its support 2 by screwing; furthermore, said threads 5′, 12′ comprise truncated protruding ends, as described above, notably with reference to FIG. 2.

As illustrated in FIG. 5, at the end of screwing, the plane annular bearing surface 13″ of the rod head 10 comes into plane contact with the bearing surface 6″ of the support 2; moreover, the base 16 of said rod head 10 is completely integrated in the non-threaded part 20 of the support 2 (in this position, the upper part of the rod head 10 makes flush with the upper face 2′ of the support 2).

In another possible embodiment, the surfaces 6″, 13″ for plane contact between the rod 4 and the support 2 may be provided under the complementary threaded parts 5, 12. In this case, the bearing surface 6″ of the support 2 is advantageously provided at the exit of the threaded part 5.

1. Osteosynthesis device comprising:
a support provided with at least one through-orifice, at least one part of the height of which is threaded, said threaded part, of axis, nominal diameter, flanks diameter and core diameter, being associated, at the entrance or the exit thereof, with a bearing surface, and
an anchoring rod of longitudinal axis, said rod having a head extended by a body adapted to anchor into the receiving bone material, wherein said head comprises a screw threaded part and a bearing surface, said threaded part having a nominal diameter, a flanks diameter an outside diameter and a root diameter, the thread of said threaded part being complementary to that of said support orifice so as to hold said rod on said support by screwing, and said bearing surfaces of the support and the rod head being complementary to one another and arranged so as to come into contact with one another when said complementary threads cooperate with each other,
wherein the protruding ends of said screw threads and support threads are truncated so that:
the difference between the outside screw diameter and the flank diameter is between 5 and 70% of the difference between the nominal diameter and the flank diameter, and
the difference between the flank diameter and the core diameter is between 5 and 70% of the difference between the flank diameter and the root diameter.

2. Osteosynthesis device according to claim 1, wherein:
the difference between the outside screw diameter and the flank diameter is between 15 and 50% of the difference between the nominal diameter and the flank diameter, and
the difference between the flank diameter and the core diameter is between 8 and 50% of the difference between the flank diameter and the root diameter.

3. Osteosynthesis device according to claim 2, wherein:
the difference between the outside screw diameter and the flank diameter is about 20% of the difference between the nominal diameter and the flank diameter, and
the difference between the flank diameter and the core diameter is about 10% of the difference between the flank diameter and the root diameter.

4. Osteosynthesis device according to claim 1, wherein it comprises a double thread.

5. Osteosynthesis device according to claim 1, wherein it comprises a triple thread.

6. Osteosynthesis device according to claim 1, wherein it comprises a thread having more than three threads.

7. Osteosynthesis device according to claim 1, wherein the bearing surfaces of the support and of the rod head are in the form of a truncated sphere, in that said truncated-sphere bearing surface of the support is provided at the exit of the orifice threaded part, and in that said truncated-sphere bearing surface of the rod head is provided under the screw threaded part in the continuation of the latter.

8. Osteosynthesis device according to claim 7, wherein the through-orifice of the support comprises a threaded upper part, located above a non-threaded lower part defining the truncated-sphere bearing surface.

9. Osteosynthesis device according to claim 1, wherein the bearing surfaces of the support and of the rod head are in the form of a truncated cone, in that said truncated-cone bearing surface of the support is provided above the orifice threaded part, and in that said truncated-cone bearing surface of the rod head is provided above the screw threaded part.

10. Osteosynthesis device according to claim 9, wherein the through-orifice of the support comprises a non-threaded upper part, located above a threaded part, said orifice upper part comprising the truncated-cone bearing surface and ensuring the complete integration of the upper part of the rod, once the latter correctly anchored in the receiving bone material.

11. Osteosynthesis device according to claim 9, wherein the support comprises a reentrant between the truncated-cone bearing surface and the threaded part of the orifice, said re-entrant being in the form of a spherical bearing surface centred on the axis of the orifice, said orifice being then adapted to receive, in replacement of said mono-axial locking anchoring rod, a poly-axial screw whose head comprises a spherical bearing that is complementary to said support spherical bearing.

12. Osteosynthesis device according to claim 1, wherein the bearing surfaces of the support and the head rod are plane surfaces, in that said plane bearing surface of the support is provided at the entrance of the orifice threaded part, and in that said plane bearing surface of the rod head is provided above the screw threaded part, said plane bearing surface of the rod head defining the lower face of a head upper base.

13. Osteosynthesis device according to claim 12, wherein the through-orifice of the support comprises a non-threaded upper part, located above the threaded part and separated from the latter by the plane bearing surface, said orifice upper part ensuring the complete integration of the upper base of the head of the rod, once the latter correctly anchored in the receiving bone material.

14. Osteosynthesis device according to claim 1, wherein the support consists of a metal plate having a thickness between 1 and 5 mm.
